(19) United States
(12) Patent Application Publication
(10) Pub. No.: US 2003/0036085 A1
(43) Pub. Date: Feb. 20, 2003

(54) Membranes

(76) Inventors: Richard F Salinaro, Hasting on Hudson, NY (US); Isaac Rothman, Brooklyn, NY (US); Thomas C Gsell, Glen Head, NY (US)

Correspondence Address: LEYDIG VOIT & MAYER, LTD, 700 THIRTEENTH ST. NW, SUITE 300, WASHINGTON, DC, 20005-3960, US

(21) Appl. No.: 10/204,224

(22) PCT Filed: Feb. 16, 2001
(86) PCT No.: PCT/US01/04974

(51) Int. Cl. C12Q 1/68; B01D 29/05

(52) U.S. Cl. 435/6; 210/483; 210/488; 210/496; 435/4

(57) ABSTRACT

Disclosed are smooth surfaced porous membranes having one or more advantages such as low autofluorescence, thermal-cyclability, especially under humid conditions, and three-dimensional binding capacity. The membrane can be free-standing or, preferably in combination with a support as in a composite membrane. The present invention provides a composite membrane comprising a porous polymer layer disposed on a support. The present invention further provides devices such as microarray devices comprising the composite for the analysis of biomaterials such as nucleic acids.

### CROSS-REFERENCE TO RELATED PATENT APPLICATIONS

This application claims the benefit of U.S. provisional patent application Nos. 60/183,327 and 60/220,825, filed Feb. 18, 2000 and Jul. 26, 2000, respectively, which are incorporated by reference.

### TECHNICAL FIELD

The present invention relates in general to smooth surfaced substrates, e.g. membranes, and in particular, to low autofluorescent smooth surfaced porous composite membranes suitable for binding biomaterials such as nucleic acids, for example, for use in microarray applications.

### BACKGROUND OF THE INVENTION

Microarrays find use in a variety of applications including screening of biomaterials, DNA sequencing, and gene expression analysis. Microarrays generally consist of a substrate such as a surface modified glass slide or a supported nylon membrane to which an array of biomaterial is attached. In a typical microarray application, the substrate is contacted with a sample containing biomaterials, such as nucleic acids, to be analyzed. The substrate is contacted with probe molecules such as labeled nucleic acids. The labeled nucleic acids hybridize with the complementary nucleic acid in the sample. The unhybridized probe molecules are removed, for example, by washing, and the microarray is then read by a suitable signal detection device, for example, by fluorescence emission.

Some of the substrates heretofore known have one or more drawbacks. For example, they have high autofluorescence, and/or require the use of a large volume of the biological sample that can be expensive. This renders it difficult to analyze rare samples that are available only in small quantities. Some of the substrates lack uniformity, leading to variability or unreliability in the results obtained.

In addition, the preparation of some substrates can involve excessive labor and/or the use of extraneous materials, e.g., adhesives used to affix the substrate to a solid support. Such extraneous materials can unnecessarily increase the background signal, e.g. fluorescence, of the microarray and reduce the sensitivity of the analysis. Many known substrates and/or adhesives are also suitable for one-time use only. The substrates lack thermal and water resistance needed for protocols involved in multiple probing.

The foregoing shows that there exists a need for a substrate that has low autofluorescence. There further exists a need for a substrate that is relatively thin and does not require a large volume of the biological sample in order to provide accurate results and/or has thermal resistance. There further exists a need for a substrate that is free or substantially free of extraneous materials.

These and other advantages of the present invention, as well as additional inventive features, will be apparent from the description of the invention provided herein.

### SUMMARY OF THE INVENTION

Many of the foregoing needs have been fulfilled by the present invention which provides smooth surfaced substrates, e.g., porous membranes, having one or more advantages such as low autofluorescence, thermal-cyclability, and three-dimensional binding capacity. The membrane can be free-standing or, preferably be in combination with a support. Accordingly, an embodiment of the present invention provides a composite membrane comprising a porous polymer layer disposed on a support. In a preferred embodiment, the membrane has capacity for binding biomaterials in three dimensions. Preferably, the membrane can be used more than once.

The present invention further provides devices such as microarray devices comprising the composite for the analysis of biomaterials such as nucleic acids. The present invention further provides a method for detecting and/or quantifying the amount of nucleic acid present in a sample. The present invention further provides a kit for determining the biomaterial present in a sample. The present invention also provides a method for preparing the membrane, composite, or device.

While the invention has been described and disclosed below in connection with certain preferred embodiments and procedures, it is not intended to limit the invention to those specific embodiments. Rather it is intended to cover all such alternative embodiments and modifications as fall within the spirit and scope of the invention.

### BRIEF DESCRIPTION OF THE DRAWINGS

FIG. 1 depicts the autofluorescence of composite membranes in accordance with an embodiment of the present invention. The membranes comprise a porous nylon 66 layer on a polycarbonate support. The X-axis represents the thickness of the nylon 66 layer and the Y-axis represents the fluorescence in arbitrary units measured for the blue and red emission lines.

FIG. 2 depicts the 3-dimensional surface profile of a composite membrane in accordance with an embodiment of the present invention. The porous polymer layer was composed of nylon 66 and the support was composed of polycarbonate. The surface profile was determined by scanning white light interference microscopy (SWLIM). The sampling depth was 1.68 μm from the surface. The magnification was 5×.

FIG. 3 depicts the 3-dimensional surface profile of a nylon 66 membrane, BIODYNE PLUS™, available from Pall Corporation. The surface profile was determined by SWLIM. The sampling depth was 1.68 μm from the surface. The magnification was 5×.

FIG. 4a depicts the scanning electron micrograph (SEM) of the bottom surface of a nylon 66 layer (surface in contact with a polycarbonate support) of a composite membrane in accordance with an embodiment of the invention. FIGS. 4b-c depict the SEMs of the top surface and the cross-section of the nylon 66 layer, respectively. FIGS. 4a-c show that the nylon 66 layer has an asymmetric structure.

### SPECIFIC DESCRIPTION OF THE INVENTION

The present invention provides a porous membrane that has a smooth surface. In accordance with an embodiment, the present invention provides a composite membrane comprising a thin porous polymer layer disposed on a support. The membranes of the present invention are attractive for use in a wide variety of applications including microarray applications.

The membrane can find use in preparing a variety of microarrays, for example, microarrays having biomaterial bound regions having a density of at least about 100 per cm², and preferably at least about 1,000 per cm², more preferably from about 1,000 per cm² to about 10,000 per cm². The regions in a microarray can have dimensions, e.g. diameters, in the range of from about 10 μm to about 250 μm, and are separated, if desired, from other regions in the array by about the same distance.

The porous polymer layer preferably includes a polymer having affinity for a biomaterial. The biomaterial can be a natural material or synthetic material. Examples of biomaterials include nucleic acids, e.g., DNA, cDNA, RNA, and mRNA, as well as at least one of oligodeoxyribonucleotides, oligoribonucleotides, antigens, proteins, peptides, lipids, lipoproteins, and polysaccharides, and derivatives thereof.

The surface of the membrane is smooth. Smoothness refers to the surface profile including peaks and valley. For example, smoother membranes have fewer peaks and valleys or less variability in the heights or the peaks and/or the depths of the valleys. Smoothness can be expressed quantitatively by any number of ways known to those skilled in the art. As an example, smoothness can be expressed in terms of the surface roughness average (Ra), the root mean square roughness (Rq), the maximum height of the profile (Rt), or the average maximum height of the profile (Rz). Ra is the mean height calculated over the entire membrane. In the measurement of Ra the effects of single spurious peaks are averaged out. Rq is the root mean square average of the measured height deviations taken within an evaluation area and measured from the mean linear surface. If a surface has a profile that contains no large deviations from the mean surface level, the values of Ra and Rq will be similar. If there are appreciable numbers of large bumps or holes, the largest values of the profile height function will dominate the surface characteristics and Rq will be larger than Ra. Rz is the average of the greatest peak-to-valley separations. Smoothness can be measured by methods known to those skilled in the art, e.g., by optical profilometry. Instruments such as, but not limited to, the WYKO™ Profilers available from Veeco Instruments Inc., Plainview, N.Y. can be employed to measure smoothness.

In accordance with one embodiment, the present invention provides a membrane having a Ra of less than about 800 nm, preferably less than about 400 nm, and more preferably less than about 200 nm. In another embodiment, the present invention provides a membrane having a Rq of less than about 1000 nm, preferably less than about 500 nm, and more preferably less than about 300 run. In yet another embodiment, the present invention provides a membrane having a Rz of less than about 10 μm, preferably less than about 8 μm, and more preferably less than about 5 μm. In still another embodiment, the present invention provides a membrane having a Rt of less than about 15 μm, preferably less than about 10 μm, and more preferably less than about 7 μm. In accordance with another embodiment of the invention, the membrane has any combination of at least two of the surface features described above.

The composite membrane of the present invention provides low or minimal signal characteristic of the polymer layer such as autofluorescence, gamma ray, X-ray, UV, VIS, or IR spectral absorbance or transmittance. For example, the composite membrane provides low autofluorescence, particularly in the visible range of the spectrum.

The autofluorescence of the membrane can be determined by any suitable method, e.g., by fluorescence detection, by confocal microscopy, or by the use of a CCD camera. For example, in fluorescence detection, a sample whose autofluorescence is to be determined is illuminated by a beam of light. The beam is red (e.g., having a wavelength of 635 nm) for a red-excited fluorescence scan, or blue (e.g. having a wavelength of 450 nm) for a blue-excited fluorescence scan. When a blue or red light hits an area of the sample containing an appropriate fluorophore, e.g. fluorochrome, the fluorophore emits light with a characteristic spectrum. The emitted light is collected and converted to a signal, e.g., by the use of a photomultiplier tube (PMT). The strength of the signal is proportional to the amount of fluorescence present in the sample. An example of a commercial system which may be used to measure the autofluorescence of the membrane includes the STORM™ optical scanner, e.g., STORM 860, by Molecular Dynamics, Sunnyvale, Calif.

The composite membrane of the present invention, in one embodiment, has an autofluorescence of less than about 5000 units, preferably less than 3000 units, more preferably less than 2000 units, and even more preferably less than 1000 units, in the wavelength range of 400-700 nm. In another embodiment, the present invention provides a composite membrane having an autofluorescence of less than about 5000 units, preferably less than 3000 units, more preferably less than 2000 units, and even more preferably less than 1000 units, in the wavelength range of 420-490 nm, particularly in the blue wavelength region of about 470 nm. In yet another embodiment. the present invention provides a composite membrane having an autofluorescence of less than about 2000 units, preferably less than 1000 units, and more preferably less than 500 units, in the wavelength range of 640-700 nm, particularly in the red wavelength region of about 650 nm. The autofluorescence units referred to herein are as measured on a STORM 860 Imager at a PMT setting of 800 V and a scanning pixel size of 100 μm, although other methods and systems can be used to measure autofluorescence as discussed above.

According to another embodiment, the present invention provides a composite comprising a porous polymer layer disposed on a support, the composite having an autofluorescence less than that of the support. For example, the autofluorescence of the composite is about 10 times, and preferably 5 times, less than that of the support. According to yet another embodiment, the present invention provides a composite comprising a porous polymer layer disposed on a support, the composite being thermal-cyclable.

The porous polymer layer is relatively thin. The porous polymer layer has a thickness of less than about 150 μm, for example, typically from about 0.1 μm to about 100 μm, preferably from about 10 μm to about 100 μm, and more preferably from about 10 μm to about 60 μm. In embodiments, the preferred thickness is from about 1 μm to about 60 μm. The membrane is preferably microporous. The polymer layer advantageously has a porosity of at least about 50% by volume, and preferably about 70% by volume or greater, and more preferably about 90% by volume. In embodiments, the membrane contains interconnected pores. The polymer layer in some embodiments, particularly those including a crystallizable polymer, have a molecular orientation in one, two, or three dimensions.

In embodiments of the present invention. The porous polymer layer has molecular characteristics, e.g., entangled macromolecular network, sufficient mechanical strength, or other characteristic(s), which make(s) the layer maintain its integrity or shape even in the absence of a support. In this respect, the porous polymer layer is capable of being a self-supporting or free-standing layer.

In embodiments, the polymer layer is of low degree of crystallinity, typically, less than about 40%, e.g., from about 10% to about 40%, preferably from about 10% to about 30%, and more preferably from about 20% to about 30%. For example, nylon 66 can be spin coated (at 5,000 rpm, 30 seconds) to produce a polymer layer of a degree of crystallinity of 24.8% (spin coating is described below). The degree of crystallinity can be determined by any suitable method, for example, by wide angle X-ray diffractometry. Higher spinning rates during coating can produce a polymer layer having a reduced degree of crystallinity.

The polymer layer is preferably porous. The porous polymer layer can typically have an average pore size of greater than about 0.01 μm, e.g. from about 0.01 μm to about 10 μm, preferably from about 0.1 μm to about 1 μm, and more preferably from about 0.2 μm to about 0.5 μm. In embodiments, the porous polymer layer has an average pore size of about 0.5 μm or more, e.g., about 0.5 μm to about 1 μm.

The pore size can be determined by any suitable method, e.g., by bubble point-porometer method. Thus, a composite membrane in an embodiment comprises a nylon 66 layer having a minimum pore size of 0.25 μm, an average flow pore size of 0.44 μm, and a bubble point pore size of 0.74 μm. The highly porous nature of the membrane makes it advantageously possible for the biomaterial to access or bind with substantially all available polymer in the thickness direction of the layer. The polymer layer preferably has affinity for the biomaterial in three dimensions. The three-dimensional affinity provides high binding capacity, e.g., higher binding capacity than substrates having two-dimensional affinity such as those that bind mainly on the surface. The three-dimensional affinity also provides the advantage that the membrane confines the fluid spotted on the membrane, e.g., in a microarray application, to a small area. The fluid does not travel with substantially different lateral velocities in different directions. As a result, it is possible to achieve highly uniform spot geometry.

In an embodiment, the porosity of the porous polymer layer is substantially, and preferably completely, random. The pore distribution is three dimensional. In embodiments, the porous polymer layer is substantially free of periodicity, e.g. it is free of regularly or systematically alternating structures. The porous regions and non-porous regions are distributed and sized randomly.

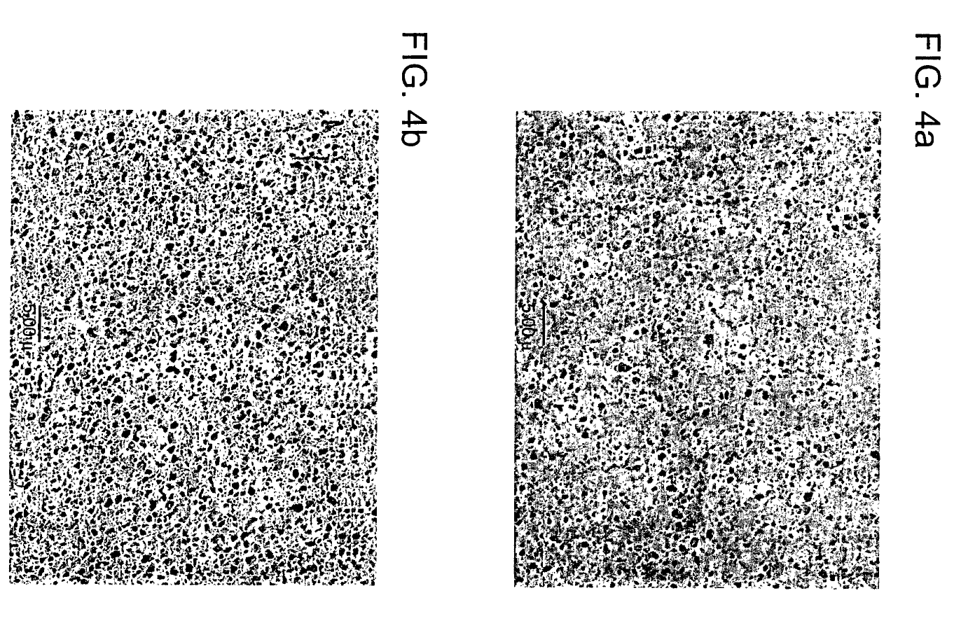

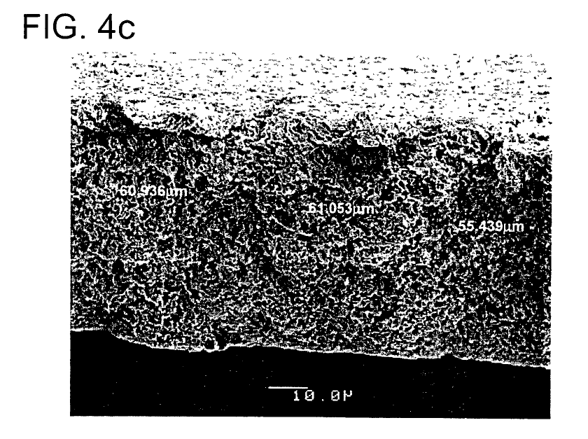

The porous polymer layer, in embodiments, have an asymmetric structure. For example, the number or the size of pores is smaller at the top surface than at the bottom surface (i.e., the surface in contact with the support). FIGS. 4a-c depict the asymmetric structure of a nylon 66 membrane. The nylon 66 membrane has a partly closed pore structure.

The polymer layer typically has a high surface area, for example, the surface area of a spin coated polymer layer is about 30%, preferably from about 30% to about 70%. and more preferably about 50%, greater than that of a polymer layer produced by casting techniques involving the use of a doctor blade. Surface area can be determined by any suitable method, e.g., the BET method. For example, nylon 66 can be spin coated to produce a layer having a BET surface area of 19.1 m²/g. The high surface area of the membrane produces low background signal and provides an advantage that it is possible to achieve high sensitivities in the detection of biomaterials. In an embodiment, a spin coated nylon 66 membrane has high sensitivity and it is possible to detect low levels of biomaterials, e.g., levels that are about 10 or more times smaller than is possible with the use of a nylon 66 membrane produced by knife casting, particularly when a label such as a red or far red fluorescing label is employed.

The polymer layer can include any suitable polymer, e.g., a polymer that has affinity for a biomaterial. The polymer layer can include a hydrophilic polymer, a polar polymer, or a charged polymer. Hydrophilic polymers include those having an affinity for water or water based solutions or a critical wetting surface tension greater than about 0.37 (±0.005) erg/sq. mm, for example, from about 0.37 to about 1 erg/sq. mm or more. and in some embodiments, from about 0.38 to about 0.73 erg/sq. mm. Polar polymers include those having a solubility parameter greater than about 10.0 (±0.2) [cal/cm³]^{1/2}, for example, from about 10 to about 20 [cal/cm³]^{1/2} or more, and preferably 10 to about 16 [cal/cm³]^{1/2}. Charged polymers include those containing positive, negative, or amphoteric polymers.

Examples of suitable polymers include a polyamide, polysulfone, polyolefin, polyhalogenated olefin, polystyrene, polyol, polyamine, polyimine, polyester, an acrylic polymer, polyacrylic acid, polyacrylic ester, polyhydroxyalkyl acrylate, polyacrylic amide, polyacrylonitrile, polyvinyl heterocyclic, polyheterocyclic, polycarbonate, polyimide, polyamide-imide, polylactide, polyglycolide, polyglycolide/lactide, polypeptide, polysiloxane, polysilane, polyacetylene, polyphosphazene, polysaccharide, polyether, epoxy resin, polyacetal, polyurethane, polyurea, urea-formaldehyde resin, polyphenol, phenol-formaldehyde resin, alkyd resin, melamine-formaldehyde resin, a dendrimer, a spiro polymer, polyaryleneoxide, polysulfide, polyketone, polyetherketone, polyetheretherketone, polyaromatic, polyaldehyde, allyl resin, cellulose, cellulose ester, cellulose derivative, and combinations thereof, wherein any of polymers, including the neutral polymers, above are modified to include a charged, polar, or hydrophilic group. An example of a polyhalogenated olefin is polyvinylidene fluoride.

For example, blends of two or more of the above polymers can be employed, and copolymers comprising monomer segments of one or more of these polymers can be employed. Examples of cellulose derivatives include ethyl cellulose, hydroxyethyl cellulose, and hydroxypropyl cellulose. Examples of cellulose esters include the nitrates, acetates, propionates, and butyrates of cellulose.

Examples of charged polymers include a polyamide, polyamine, polyimine, polyacrylic amide, polyvinyl heterocyclic, polyheterocyclic, polyimide, polyamide-imide, polypeptide, polyurethane, polyurea, urea-formaldehyde resin, melamine-formaldehyde resin, a dendrimer, and cellulose derivatives.

Examples of hydrophilic polymers include a polyamide, polyol, polyamine, polyimine, polyester, an acrylic polymer, polyacrylic acid, polyacrylic ester, polyhydroxyalkyl acrylate, polyacrylic amide, polyacrylic nitrile, polyvinyl heterocyclic, polyheterocyclic, polyimide, polyamide-imide, polylactide, polypeptide, polysaccharide, polyether, epoxy resin, polyacetal, polyurethane, polyurea, urea-formaldehyde resin, polyphenol, phenol-formaldehyde resin, alkyd resin, melamine-formaldehyde resin, a dendrimer, a spiro polymer, polysulfide, polyketone, polyaldehyde, cellulose, cellulose ester, or cellulose derivative; a polysulfone, polyolefin, polyhalogenated olefin, polystyrene polycarbonate, polysiloxane, polysilane, polyacetylene, polyphosphazene, polyaromatic, polyaryleneoxide, allyl resin, polyetheretherketone, and polyetherketone, which may be hydrophilic as such as or which have been modified to be hydrophilic; and combinations thereof.

Particular examples of polar polymers include a polyamide, polysulfone, polyol, polyamine, polyimine, polyester, an acrylic polymer, polyacrylic acid, polyacrylic ester, polyhydroxyalkyl acrylate, polyacrylic amide, polyacrylic nitrile, polyvinyl heterocyclic, polyheterocyclic, polycarbonate, polyimide, polyamide-imide, polylactide, polypeptide, polysaccharide, polyether, epoxy resin, polyacetal, polyurethane, polyurea, urea-formaldehyde resin, polyphenol, phenol-formaldehyde resin, alkyd resin, melamine-formaldehyde resin, a dendrimer, a spiro polymer, polyaryleneoxide, polysulfide, polyketone, polyetherketone, polyetheretherketone, polyaromatic, polyaldehyde, cellulose, cellulose ester, or cellulose derivative; a polyolefin, polyhalogenated olefin, polysiloxane, polyacetylene, polyphosphazene, polystyrene, polysilane, and allyl resin, as such or which have been modified to render them polar; and combinations thereof. To render non-polar polymers polar, any suitable method can be used, e.g., oxidation, reduction, sulfonation, nitration, amidation, carbonylation, hydroxylation, carboxylation, phosphorylation, treatment with surfactants, and/or grafting of polar monomers or polymers.

Preferably, the polymer layer includes a polyamide, copolyamide, polysulfone, or polyvinylidene fluoride. Particular examples of polyamides and copolyamides include nylons, e.g., nylon 4, nylon 45, nylon 6, nylon 66, nylon 11, nylon 610, nylon 612, and nylon 6T. Nylon 66 is a further preferred polyamide. Particular examples of polysulfone include bisphenol A polysulfone, polyethersulfone, and polyarylsulfone. Polyethersulfone is a further preferred polysulfone.

One or more polymers described above can be cast, coated, or applied as a porous polymer layer. Alternatively, one or more polymers can be combined with a modifying polymer and cast, coated, or applied as a porous layer. The modifying polymer may be associated by any number of mechanisms, e.g., covalent, ionic, coordinate, hydrogen bonding, or van der waals interactions or bonding, with the porous polymer such that it imparts hydrophilicity, charge, or polarity to the porous layer. In another embodiment, a porous layer can be first formed, and the surface modified by coating and/or curing a modifying polymer or mixture of polymers or polymerizing a modifying monomer or mixture of monomers.

The modifying polymer or monomer can have a variety of functional groups, e.g. hydroxyl, carboxyl, amine, pyridyl, sulfonic, sulfhydryl, thiocarbonyl, phosphine, phosphoryl, and imine. Examples of suitable modifying polymers include polyethyleneimine, polyvinyl alcohol, polyacrylic acid, hydrolyzed copolymer of maleic anhydride/methylvinyl ether, polystyrene sulfonic acid or a salt thereof, polyvinyl sulfonic acid or a salt thereof, as described in, e.g., U.S. Pat. No. 4,707,266; hydroxyl containing polymers such as polyhydroxyacrylates, as described in, e.g., U.S. Pat. Nos. 4,959,150; 4,906,374; 4,964,989; 5,019,260; and 4,886,836; and cationic polyamido/polyamino-epichlorohydrin resins and polyamine epichlorohydrin resins, as described in, e.g., U.S. Pat. Nos. 4,702,840 and 5,128,041.

The membrane of the present invention is preferably hydrophilic. The membrane is wettable by water, aqueous liquids, and/or organic solvents, particularly polar organic solvents. The critical wetting surface tension of the membrane surface can be at any suitable value, typically about 0.4 erg/sq. mm or greater, e.g., from about 0.72 erg/sq. mm to about 1 erg/sq. mm, and preferably from about 0.72 erg/sq. mm to about 0.90 erg/sq. mm, at 25° C.

The support of the composite membrane can be porous, or more preferably, can be non-porous. The support can have a suitable shape, e.g., planar, rod, spherical, or elliptical, preferably a sheet, for example, in the form of a slide or disc. The sheet support can have a suitable thickness, typically a thickness greater than 0.01 mm, e.g., from about 0.01 mm to about 2 mm, and preferably from about 0.11 mm to about 1 mm. The support should have sufficient mechanical strength, e.g., flexural strength, to withstand the stresses involved in fabricating the composite or microarray device as well as the stresses involved during use, e.g., during hybridization, immunoassay, Western blotting, and/or analysis (including automated analysis).

The support can include any suitable material, preferably a low signaling, particularly a low fluorescing material. The support can be organic or inorganic. Inorganic supports include glass, ceramics, gels, or metals, and organic supports include a support polymer or polymeric gel. The support can optionally include a reflective coating on one or both sides. Examples of reflective coatings include metal coatings. Thus, the composite membrane can include a reflective coating between the porous polymer layer and the support. The reflective coating can reduce or eliminate the autofluorescence of the support material. The reflective coating can be provided by methods known to those skilled in the art, e.g., by sputtering or vacuum evaporation.

The organic support can include, e.g., a polyamide, polysulfone, polyolefin, polyhalogenated olefin, polystyrene, polyol, polyamine, polyimine, polyester, an acrylic polymer, polyacrylic acid, polyacrylic ester, polyhydroxyalkyl acrylate, polyacrylic amide, polyacrylonitrile, polyvinyl heterocyclic, polyheterocyclic, polycarbonate, polyimide, polyamide-imide, polylactide, polypeptide, polysiloxane, polysilane, polyacetylene, polyphosphazene, polysaccharide, polyether, epoxy resin, polyacetal, polyurethane, polyurea, urea-formaldehyde resin, polyphenol, phenol-formaldehyde resin, alkyd resin, melamine-formaldehyde resin, a dendrimer, a spiro polymer, polyaryleneoxide, polysulfide, polyketone, polyetherketone, polyetheretherketone. polyaromatic, polyaldehyde, allyl resin, cellulose, cellulose ester, cellulose derivative, and combinations thereof.

Particular examples of supports include glass, acrylic, and polycarbonate. A particular support material comprises a polyolefin resin such as TOPAS™ resin. In embodiments of the present invention, the composite is substantially free of an adhesion promoter between the polymer layer and the support. If desired, the composite can include an adhesion promoter between the polymer layer and the support. For example, an adhesion promoter may be desirable to improve the bonding between glass and certain polymer layers such as polyamide. The adhesion promoter should not contribute to or otherwise interfere with the detection and/or measurement of the fluorescence or other signal of the composite membrane.

Any suitable adhesion promoter can be used, e.g., a coupling agent such as a silane coupling agent can be used. Alternatively, an adhesion promoter such as a titanate can be used. Examples of suitable silane coupling agents include aminosilanes, halosilanes, alkoxysilanes, and glycidylsilanes. Examples of suitable titanates include titanium alkoxides, titanium aminoalkoxides, titanium alkoxide alkanedionates, and titanium ammonium lactates, and preferably titanium alkoxides and titanium aminoalkoxides.

Alternatively, or in addition, the substrate can be surface treated, e.g., surface-treated, before placing the polymer layer on the substrate, by a suitable method, e.g., plasma treatment, corona discharge, flaming, oxidation, sputtering, etching, and the like.

According to an embodiment of the present invention, the composite can be charged, e.g., positively or negatively charged. The positively charged composite can include, for example, a quaternary ammonium group. Thus, for example, a positively charged polymer can be disposed on the support, or a coating of a charged polymer can be placed on the uncharged polymer layer. The negatively charged composite can include, for example, a sulfonic or carboxylic group. In accordance with another embodiment, the polymer layer can include a polar group.

The composite membrane of the present invention has an advantage that it has thermal cycling resistance, particularly under humid conditions, as, for example, encountered during hybridization. The composite does not disintegrate or delaminate under the humid conditions. Thus, in some embodiments of the invention, the composite can be used more than once. This property makes the membrane attractive for multiple probing, for example, hybridizing and probing the same array two, three, or more times. During multiple probing, the composite membrane allows for the removal of the previously bound probe without affecting the biomaterial bound to the membrane.

Embodiments of the present invention include a composite comprising a planar charged polymer layer disposed on a support, wherein the planar charged polymer layer is porous and has a Ra of less than about 800 nm, a composite comprising a planar charged polymer layer disposed on a support, wherein the planar charged polymer layer is porous and has a Rq of less than about 1000 nm, a composite comprising a planar charged polymer layer disposed on a support, wherein the planar charged polymer layer is porous and has a Rz of less than about 10 μm, and a composite comprising a planar charged polymer layer disposed on a support, wherein the planar charged polymer layer is porous and has a Rt of less than about 15 μm.

Embodiments of the present invention also include a composite comprising a planar hydrophilic polymer layer disposed on a support, wherein the planar hydrophilic polymer layer is porous and has a Ra of less than about 800 nm, a composite comprising a planar hydrophilic polymer layer disposed on a support, wherein the planar hydrophilic polymer layer is porous and has a Rq of less than about 1000 nm, a composite comprising a planar hydrophilic polymer layer disposed on a support, wherein the planar hydrophilic polymer layer is porous and has a Rz of less than about 10 μm, a composite comprising a planar hydrophilic polymer layer disposed on a support, wherein the planar hydrophilic polymer layer is porous and has a Rt of less than about 15 μm, a composite comprising a planar hydrophilic charged polymer layer disposed on a support, wherein the planar hydrophilic charged polymer layer is porous and has a Ra of less than about 800 nm, a composite comprising a planar hydrophilic charged polymer layer disposed on a support, wherein the planar hydrophilic charged polymer layer is porous and has a Rq of less than about 1000 nm, a composite comprising a planar hydrophilic charged polymer layer disposed on a support, wherein the planar hydrophilic charged polymer layer is porous and has a Rz of less than about 10 μm, a composite comprising a planar hydrophilic charged polymer layer disposed on a support, wherein the planar hydrophilic charged polymer layer is porous and has a Rt of less than about 15 μm, a composite comprising a planar polar polymer layer disposed on a support, wherein the planar polar polymer layer is porous and has a Ra of less than about 800 nm, a composite comprising a planar polar polymer layer disposed on a support, wherein the planar polar polymer layer is porous and has a Rq of less than about 1000 nm, a composite comprising a planar polar polymer layer disposed on a support, wherein the planar polar polymer layer is porous and has a Rz of less than about 10 μm, and a composite comprising a planar polar polymer layer disposed on a support, wherein the planar polar polymer layer is porous and has a Rt of less than about 15 μm.

Other embodiments of the present invention include a porous membrane comprising a smooth planar hydrophilic polymer surface having a Ra of less than about 800 nm, a porous membrane comprising a smooth planar hydrophilic polymer surface having a Rq of less than about 1000 nm, a porous membrane comprising a smooth planar hydrophilic polymer surface having a Rz of less than about 10 μm, a porous membrane comprising a smooth planar hydrophilic polymer surface having a Rt of less than about 15 μm, a porous membrane comprising a smooth planar charged polymer surface having a Ra of less than about 800 nm, a porous membrane comprising a smooth planar charged polymer surface having a Rq of less than about 1000 nm, a porous membrane comprising a smooth planar charged polymer surface having a Rz of less than about 10 μm, a porous membrane comprising a smooth planar polar polymer surface having a Rt of less than about 15 μm, a porous membrane comprising a smooth planar polar polymer surface having a Ra of less than about 800 nm, a porous membrane comprising a smooth planar polar polymer surface having a Rq of less than about 1000 nm, a porous membrane comprising a smooth planar polar polymer surface having a Rz of less than about 10 μm, and a porous membrane comprising a smooth planar polar polymer surface having a Rt of less than about 15 μm.

Embodiments of the present invention also include composites which have low autofluorescence and/or are thermal-cyclable. Such embodiments include a composite comprising a porous charged polymer layer disposed on a support and a composite comprising a porous polyamide layer disposed on a support. Further such embodiments include a composite comprising a porous hydrophilic polymer layer disposed on a support, a composite comprising a porous hydrophilic charged polymer layer disposed on a support, and a composite comprising a porous polar polymer layer disposed on a support.

Still further embodiments of the present invention include composites having autofluorescence less than that of their support. Such embodiments include a composite comprising a porous charged polymer layer disposed on a support, a composite comprising a porous polyamide layer disposed on a support, a composite comprising a porous hydrophilic polymer layer disposed on a support, and a composite comprising a porous polar polymer layer disposed on a support.

The membranes of the present invention can be prepared by methods known to those skilled in the art, for example, by a method involving phase inversion, such as a thermally induced phase inversion, polymer induced phase inversion, or non-solvent induced phase inversion, of a polymer solution or melt which has been cast or extruded in the form a thin sheet on a support. Certain embodiments of membranes may be produced by vacuum deposition such as chemical or physical vapor deposition, sputtering, and anodic arc deposition, plasma polymerization, interfacial polycondensation, or Langmuir-Blodgett techniques. The polymer solution can be cast on a support by knife coating, blade coating, dip coating, rod coating, roll coating, gravure coating, slot coating, slide coating, screen printing, or preferably spin coating.

In a preferred embodiment, membranes of the present invention are prepared by spin coating a polymer solution on a support followed by causing phase invention by contacting with a non-solvent. The porosity of the membrane can be controlled by providing a suitable time between the spin coating and the non-solvent contacting. Longer time intervals tend to produce tighter membranes, e.g., with smaller pore size. In embodiments of the invention, the support is contacted with the non-solvent immediately after the spin coating.

Accordingly, the present invention provides a method for preparing the composite membranes described above, the method comprising (a) providing a support; (b) providing a composition comprising a solvent and the polymer of the polymer layer; (c) spin coating the support with the composition in (b); (d) removing the solvent; and (e) recovering the composite membrane.

The polymer of the polymer layer can be dissolved in any suitable solvent.

Examples of suitable solvents for polyamides include formic acid and dimethyl acetamide. See, e.g., U.S. Pat. No. 4,340,479. Examples of suitable solvents for polysulfones include dimethyl formamide and dimethyl acetamide. Examples of suitable solvents for polyvinylidene fluoride include triethylphosphite and dimethyl formamide. Additional examples of solvents can be found in the literature, e.g., R. E. Kesting, Synthetic Polymer Membranes, pp. 186-223 (1985). The polymer solution may also contain suitable pore formers such as water, polyvinyl pyrrolidone, formamide, dimethylsulfoxide, zinc chloride, and glycerol.

In a preferred embodiment, the polymer solution is placed on a support which rests on a driven platform. The speed of the platform, or the spinning speed, can be as high as, for example, 10,000 rpm or more, typically from about 1000 rpm to about 10,000 rpm, and preferably from about 2,000 rpm to about 8,000 rpm. It is believed that the centrifugal force forces the polymer solution over the support leaving a thin, uniform film behind. In addition, during the coating the process some of the solvent may be removed as a result of evaporation. This can increase the viscosity of the polymer solution leading to the formation of a level thin coating. The coating process can be carried out in a controlled atmosphere where temperature and/or humidity can be controlled, e.g., to adjust the porosity of the membrane.

The spin coated support can be immersed in a non-solvent bath to coagulate the polymer layer. Examples of non-solvents include water, alcohol, and mixtures containing one or more of water, formamide, formic acid, and alcohol. The resulting composite membrane is typically dried to remove some or all of the non-solvent associated with the membrane. If desired, the membrane can be separated from the support. For microarray applications, the membrane is preferably employed in a composite form. The membrane provides a sharp spot geometry in association with biomaterials.

As discussed above, polymer blends also can be used to cast the membrane of the present invention. Preferably, the polymer blend comprises at least one polar, hydrophilic, or charged polymer. Immiscible or partially miscible blends will phase separate as the membrane film solidifies. A porous structure can be created by selectively removing one of the polymer phases, e.g., by extracting with a solvent. applying heat, or etching. Membranes prepared by this approach can result in a porous network having pores smaller than the wavelength of light involved in fluorescence studies. Membranes, especially those having a thickness of about 0.1 μm to about 0.4 μm, will have reduced reflectivity to light. Preferred embodiments of the membranes will be transparent to incident light, and the autofluorescence will be minimal or eliminated. Embodiments of the membrane have low light reflectivity and are transparent.

Accordingly, the present invention provides a method for preparing a composite having low autofluorescence comprising a porous charged, hydrophilic, or polar polymer layer disposed on a support, the method comprising (a) providing a support; (b) providing a composition comprising a solvent and a plurality of polymers at least one of which is a charged, hydrophilic, or polar polymer; (c) spin-coating the support with the composition from (b); (d) removing the solvent; (e) selectively removing one or more polymers so as leave behind the charged, hydrophilic, or polar polymer; and (f) recovering the composite.

The present invention further provides a method for identifying or measuring the amount of a biomaterial, particularly nucleic acid, present in a sample. The method comprises (a) providing a composite membrane as described above; (b) contacting the sample with the composite membrane to obtain a biomaterial bound composite; (c) contacting the biomaterial bound composite membrane with a binding agent such as a nucleic acid probe that is capable of hybridizing or associating with the biomaterial; and (d) determining whether hybridization or association has occurred. Embodiments of the method also include sandwich assays.

In performing the assays of some embodiments of the present invention, a sample containing the biomaterial to be assayed is contacted with the membrane under conditions suitable for binding the biomaterial to the membrane. The binding can involve any number of mechanisms, preferably through a non-covalent binding mechanism such as hydrophobic interaction. After the biomaterial is bound to the membrane, the membrane is contacted with one or more binding agents such as probe molecules so as to create association of the probe molecules with the bound biomaterials. For example, where the biomaterial of interest is a nucleic acid, the membrane is contacted with the probe molecules under hybridization conditions, e.g., in a 10% solution of sodium dodecyl sulfate solution. The present invention provides a microarray comprising the smooth or low autofluorescent composite or membrane and a plurality of biomaterial associated with the porous polymer layer of the composite or membrane. In an embodiment, the biomaterial includes nucleic acid or protein.

Any suitable hybridization condition can be employed, e.g., highly stringent or moderately stringent conditions. For example, low stringency hybridization conditions may be at 50° C. and 6× SSC (0.9 M sodium chloride/0.09 M sodium citrate) while hybridization under stringent conditions may be at 50° C. or higher and 0.1× SSC (15 mM sodium chloride/1.5 mM sodium citrate). The excess probe molecules are typically removed, and the microarray is analyzed for a signal such as fluorescence.

Any suitable binding agent such as a probe can be employed, for example, the nucleic acid probe comprises a nucleic acid and a label such as a fluorescent label. In those embodiments wherein 2 or more binding agents are utilized, at least one binding agent is preferably a specific binding agent. Suitable labels include fluorescein, rhodamine, BODIPY, cyanine dyes such as Cy5 and the like, and radioactive isotopes, such as, e.g., ³³S, ³²P, and ³H. Multiple probes and multiple labels can be utilized in accordance with embodiments of the invention.

The membranes of the present invention can be fabricated into a variety of different microarrays. For example, the microarrays can be used in the binding and hybridization of polynucleotides and analogues or mimetics thereof, including, nucleic acids in which the phosphodiester linkage has been replaced with a substitute linkage. such as phophorothioate, methylimino, methylphosphonate, phosphoramidate, guanidine and the like; nucleic acids in which the ribose subunit has been substituted, e.g., hexose phosphodiester; peptide nucleic acids; and the like.

The present invention further provides modifications of the assay described above. For example, an embodiment of the invention includes an assay in which the probe molecules are bound to the microarray or composite and the sample to be analyzed is placed in contact with the probe under hybridization or immunoassay conditions. The assay can comprise a double antibody or a sandwich assay. The complex including the biomaterial of interest and at least one labeled binding agent can be analyzed to detect a signal.

Embodiments of the invention can be carried out with a variety of techniques and assays, such as hybridization assays and immunoassays, including, but not limited to, arrays (e.g., microarrays, including but not limited to those described in “Microarrays: Biotechnology's Discovery Platform for Functional Genomics,” M. Schena, et al., Trends in Biotechnology, 16, 301-306 (1998)), mRNA abundance analyses, Southern, Northern, Western, Southern-Western, dot, slot, and colony blots. Embodiments of the invention are compatible with automated and semi-automated protocols, as well as high throughput applications.

The present invention further provides a kit for identifying or measuring the amount of a biomaterial such as a nucleic acid present in a sample comprising (a) a composite having low autofluorescence comprising a polymer layer disposed on a support; and (b) at least one binding agent such as a nucleic acid probe. The present invention further provides a device for identifying or measuring the amount of a biomaterial such as a nucleic acid present in a sample comprising a composite; wherein the device is arranged to allow a sample to contact the composite, or to contact a binding agent on the composite. After a complex is formed between the sample and at least one binding agent, the biomaterial is identified and/or quantified.

The following examples further illustrate the present invention, but of course should not be construed in any way as limiting the scope of the invention.

### EXAMPLE 1

This Example illustrates a method of preparing composite membranes in accordance with an embodiment of the present invention and their low autofluorescence.

Nylon 66 solutions, 14.5% by weight solids in a solvent containing 86% by weight formic acid and 14% by weight water, were spin coated on a polycarbonate support disc. A spin coater from Specialty Coating Systems, Inc. in Indianapolis, Ind. Model No. P6204-A was employed. The resulting coated support was immersed in a bath containing 52% formic acid solution in water at 60° C. The membrane was dried in an oven at 100° C. for about 15 minutes. Membranes of varying thicknesses were prepared by varying the spinning speed and spinning time.

The membranes were tested for autofluorescence by irradiating with laser beams. and the emission intensity in the blue and red wavelengths were measured on a STORM™ 860 Imager. The photomultiplier tube of the imager was operated at the 800 V setting and the scanning pixel size was 100 μm. The results obtained are set forth below.

| | Spin- | | | | |
|---|---|---|---|---|---|
| Membrane | ning | | Average | blue | red |
| or | speed, | Spinning | thickness, | spectrum, | spectrum, |
| support | rpm | time, sec | mil | fl. units | fl. units |
| N66 | 5000 | 30 | 0.8 | 229 ± 74 | 113 ± 89 |
| N66 | 8000 | 30 | 1.0 | 183 ± 24 | 103 ± 82 |
| N66 | 8000 | 60 | 1.1 | 180 ± 91 | 109 ± 76 |
| N66 | 2500 | 30 | 1.7 | 494 ± 160 | 185 ± 186 |
| Poly- | — | — | — | 614 ± 56 | 749 ± 126 |
| carbonate | | | | | |
| support | | | | | |
| BIODYNE | — | — | 5.2 | 4892 ± 651 | 822 ± 148 |
| PLUS | | | | | |

The foregoing shows that the membranes of the present invention have low autofluorescence.

### EXAMPLE 2

This Example illustrates the smooth surface of a composite membrane prepared in accordance with an embodiment of the present invention.

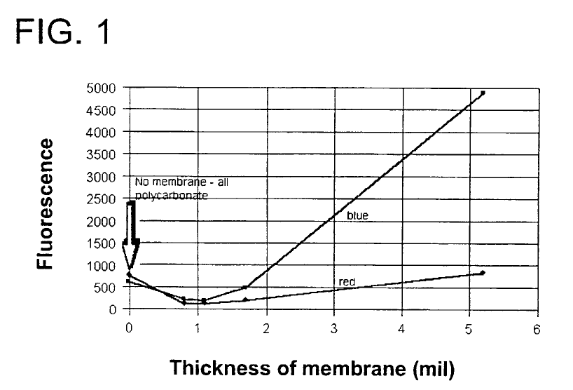

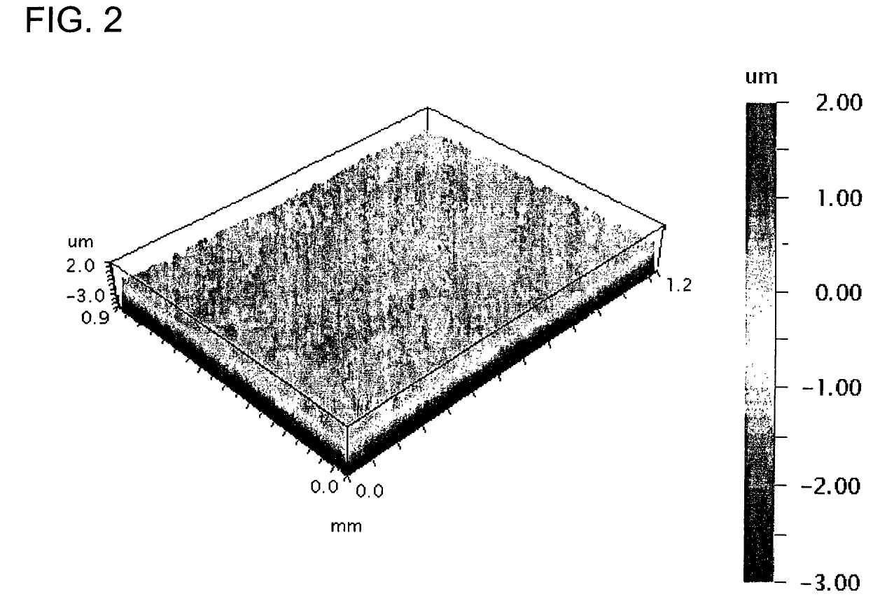

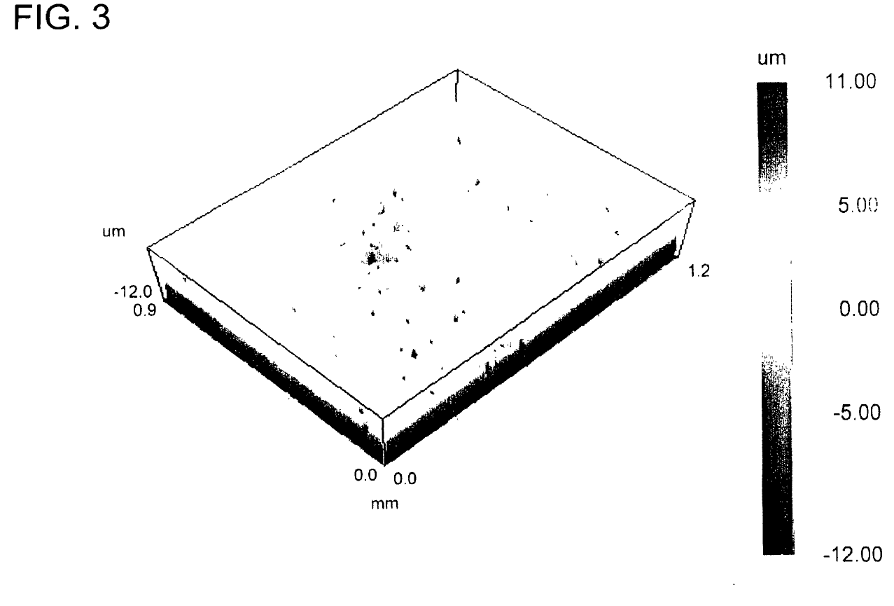

A composite membrane comprising a nylon 66 layer on a polycarbonate support was prepared and the surface was analyzed by SWLIM on a WYKO optical profiler. The 3-dimensional plot of the surface profile obtained is depicted in FIG. 1. A 3-dimensional plot of the surface profile of a comparative membrane, BIODYNE PLUS, from Pall Corporation was also obtained and the plot is depicted in FIG. 2. Ra, Rq, Rt, and Rz were measured and are set forth below for the two membranes.

| | Ra, nm | Rq, nm | Rt, μm | Rz, μm |
|---|---|---|---|---|
| Composite Membrane | 187.3 | 268.28 | 4.28 | 6.57 |
| BIODYNE PLUS | 924.7 | 1116.0 | 12.14 | 16.34 |

The foregoing shows that the composite membrane of the present invention has a smooth surface profile.

### EXAMPLE 3

This Example illustrates the thermal cyclability of a composite membrane in accordance with an embodiment of the present invention. A composite membrane comprising a spin coated nylon 66 layer on a polycarbonate support was autoclaved while submerged in a 10% sodium dodecyl sulfate solution. Two cycles of autoclaving were completed, each cycle lasting for 1 hour and the temperature of the autoclave was 118° C. This an accelerated test of the conditions involved in a typical hybridization procedure which is normally carried out at a temperature of about 45° C. overnight. At the end of two cycles, the membrane was examined visually, and no delamination of the polymer layer was observed.

### EXAMPLE 4

This Example illustrates the excellent adhesion of the polymer layer to the support. A nylon 66 layer was spin coated on a polycarbonate support. The resulting composite membrane was exposed to a 10% sodium dodecyl sulfate solution at 45° C. for a period of 6 weeks. No delamination of the polymer layer was observed.

In another test, a composite membrane was subjected to 28 autoclave cycles while immersed in a 10% sodium dodecyl sulfate solution at 120° C., each cycle lasting for a period of 30 minutes. No delamination of the polymer layer was observed.

All references cited herein, including patents and publications, are incorporated herein in their entireties by reference.

While this invention has been described with an emphasis upon several embodiments, it will be obvious to those of ordinary skill in the art that variations of the embodiments may be used and that it is intended that the invention may be practiced otherwise than as specifically described herein. Accordingly, this invention includes all modifications encompassed within the spirit and scope of the invention as defined by the following claims.

What is claimed is:

1. A composite comprising a polymer layer disposed on a support, wherein the polymer layer is porous and has at least one of (a) a surface roughness average (Ra) of less than about 800 nm, (b) a root mean square surface roughness (Rq) of less than about 1000 nm, (c) an average maximum height of the surface profile (Rz) of less than about 10 μm, and (d) a maximum height of the surface profile (Rt) of less than about 15 μm.

2. A composite comprising a polymer layer disposed on a support, the composite having low autofluorescence.

3. A composite comprising a polymer layer disposed on a support, the composite being thermal-cyclable.

4. The composite of any of claims 1-3, wherein the polymer layer comprises a polyamide.

5. The composite of any of claims 1-3, wherein the polymer layer comprises a porous hydrophilic polymer layer.

6. The composite of any of claims 1-3, wherein the polymer layer comprises a porous charged polymer layer.

7. The composite of any of claims 1-3, wherein the polymer layer comprises a polar polymer layer.

8. The composite of any of claims 1-7, having an autofluorescence of less than about 5000 units in the wavelength range of 400-700 nm.

9. The composite of claim 8, having an autofluorescence of less than about 5000 units in the wavelength range of 420-490 nm.

10. The composite of any of claims 1-7, having an autofluorescence of less than about 2000 units in the wavelength range of 640-700 nm.

11. The composite of claim 10, having an autofluorescence of less than about 2000 units in the wavelength range of 400-700 nm.

12. The composite of claim 11, having an autofluorescence of less than about 1000 units.

13. The composite of any of claims 1-12, having an autofluorescence less than that of the support.

14. The composite of claim 13, having an autofluorescence about 10 times less than that of the support.

15. The composite of claim 13 or 14, having an autofluorescence about 5 times less than that of the support.

16. The composite of any of claims 1-15, wherein the polymer or polyamide layer has a thickness of less than about 150 μm.

17. The composite of claim 16, wherein the polymer or polyamide layer has a thickness of from about 10 μm to about 100 μm.

18. The composite of any of claims 1-17, wherein the polymer or polyamide layer has affinity for a biomaterial.

19. The composite of claim 18, wherein the biomaterial includes nucleic acid.

20. The composite of claim 4, wherein the polyamide layer comprises a nylon.

21. The composite of claim 20, wherein the nylon is nylon 66.

22. The composite of any of claims 1-3 and 8-19, wherein the porous polymer layer comprises a polysulfone.

23. The composite of any of claims 1-3 and 8-19, wherein the porous polymer layer comprises polyvinylidene fluoride.

24. The composite of any of claims 1-23, wherein the support is non-porous.

25. The composite of claim 24, wherein the support comprises glass, ceramic, metal, or a support polymer.

26. The composite of claim 25, wherein the support polymer comprises polycarbonate, acrylic resin, polystyrene, or polyolefin.

27. The composite of any of claims 1-26, which is substantially free of an adhesion promoter.

28. The composite of claim 24 or 25, wherein the support is glass.

29. The composite of claim 28, which includes an adhesion promoter.

30. The composite of claim 29, wherein the adhesion promoter is a silane.

31. The composite of claim 6, wherein the charged polymer layer includes a positively charged polymer.

32. The composite of claim 6, wherein the charged polymer layer includes a positively charged coating and a neutral polymer.

33. The composite of claim 31 or 32, which includes quaternary ammonium groups.

34. A porous membrane comprising a smooth planar hydrophilic polymer surface, a smooth planar charged polymer surface, or a smooth planar polar polymer surface, the porous membrane having at least one of (a) a surface roughness average (Ra) of less than about 800 nm, (b) a root mean square surface roughness (Rq) of less than about 1000 nm, (c) an average maximum height of the surface profile (Rz) of less than about 10 μm, and (d) a maximum height of the surface profile (Rt) of less than about 15 μm.

35. A method for identifying or measuring the amount of a biomaterial present in a sample comprising (a) providing a composite or porous membrane of any of claims 1-34; (b) contacting the sample with the composite to obtain a biomaterial bound composite; (c) contacting the biomaterial bound composite with a binding agent that is capable of binding with the biomaterial; and (d) determining whether binding has occurred.

36. The method of claim 35, wherein the biomaterial includes nucleic acid.

37. The method of claim 35 or 36, wherein the binding agent includes a nucleic acid and a label.

38. A kit for identifying or measuring the amount of a biomaterial present in a sample comprising (a) a composite or porous membrane of any of claims 1-34 and (b) a biomaterial probe.

39. A device for identifying or measuring the amount of a biomaterial present in a sample comprising a composite or porous membrane of any of claims 1-34; an arrangement for contacting the sample with the composite; an arrangement for contacting the composite with a biomaterial probe; and an arrangement for determining whether binding has occurred between the biomaterial and the probe.

40. A method for preparing a composite having low autofluorescence comprising a porous polymer layer disposed on a support, the method comprising (a) providing a support; (b) providing a composition comprising a solvent and a polymer; (c) spin-coating the support with the composition from (b); (d) removing the solvent; and (e) recovering the composite.

41. The method of claim 40, wherein the polymer is a hydrophilic polymer or a charged polymer.

42. The method of claim 40 or 41, wherein the porous polymer layer comprises a polyamide.

43. A method for preparing a composite having low autofluorescence comprising a porous polymer layer disposed on a support, the method comprising (a) providing a support; (b) providing a composition comprising a solvent and a plurality of polymers at least one of which is a charged, hydrophilic, or polar polymer; (c) spin-coating the support with the composition from (b); (d) removing the solvent; (e) selectively removing one or more polymers so as to leave behind the charged, hydrophilic, or polar polymer; and (f) recovering the composite.

44. The composite prepared by the method of any of claims 40-43.
